(19) United States
(12) Patent Application Publication
(10) Pub. No.: US 2022/0132893 A1
(43) Pub. Date: May 5, 2022

(54) Process for Preparing a Product from One or More Biological Substances or Mixtures Thereof, a Product Prepared by This Process and Use of Such a Product

(71) Applicant: Luya Foods AG, Zollikofen (CH)

(72) Inventors: Christoph Denkel, Zürich (CH); Daniel Heine, Bern (CH); Michael Whyte, Chur (CH); Carlotta Sartori, Köniz (CH); Tobias Kistler, Zug (CH)

(21) Appl. No.: 17/431,016

(22) PCT Filed: Jul. 23, 2019
(86) PCT No.: PCT/EP2019/000226

§ 371 Date: Aug. 13, 2021

(30) Foreign Application Priority Data
Feb. 13, 2019 (CH) 00180/19

(51) Int. Cl. A23L 11/50; A23J 3/22; A23L 23/00; A23L 31/00; A23P 30/20

(52) U.S. Cl. CPC A23L 11/50; A23J 3/227; A23C 20/025; A23L 31/00; A23P 30/20; A23L 23/00

(57) ABSTRACT

The invention relates to a process for producing a product from one or more biological substances or mixtures thereof. Furthermore, the invention relates to a product manufactured according to the process of the invention. Furthermore, the invention relates to the use of such a product.

### CROSS-REFERENCE TO RELATED APPLICATIONS

This application is a U.S. national phase application filed under 35 U.S.C. § 371 of International Application No. PCT/EP2019/000226, filed Jul. 23, 2019, designating the United States, which claims priority to Swiss application No. 00180/19, filed Feb. 13, 2019, which are hereby incorporated herein by reference in their entirety.

The invention relates to a process for producing a product from one or more biological substances or mixtures thereof.

Furthermore, the invention relates to a product manufactured according to the process of the invention.

Furthermore, the invention relates to the use of such a product.

### STATE OF THE ART

It is known to produce structured, vegetarian or vegan meat-like products using an extrusion process, whereby in terms of composition an isotopic mass is processed by high temperatures and shear forces and broken down, in order to then be structured by application of decreasing temperatures and a shear gradient directed from the outside towards the center of the flowing mass melt, with the result being a displacement of different layers of the extrudate against each other giving rise to a fibrous structure post-cooling. Products such as these are known, for example, under the name “Beyond Meat”. The disadvantage of such extrusion processes is the high thermal load of the products being created. The composition of the product is often defined by the desired product structure. Adjustments to the recipe, which mostly contain soy, wheat or pea protein with different hydrocolloid mixtures and flavorings, for example, force extensive and expensive experiments to optimize process and product design [1].

It is known in this context to produce such products from dried protein isolates or high concentrates, which requires a high input of energy related to the process. In addition, due to the high pressures and temperatures, extrusion cooking processes are not considered to be mild on the product and therefore they cannot be used for the production of organic products in Switzerland, for example [2].

In some cases, products are also referred to or understood as meat substitutes that have only undergone a process in which a—mostly plant-based—fluid is aggregated and thus concentrated and subsequently solidified. An example of this is tofu. The disadvantage of this is that the structure of the product results from the random internal structure of the insolubilized ingredients of the fluid, whereby the structure must be described as isotropic. The firmness of the product results indirectly from the dry matter to be adjusted when pressing the precipitated material. In principle, soluble proteins, which are made insoluble, are used for structuring. All other ingredients, such as carbohydrates, fibers or insoluble proteins, are usually not used, so that a product side stream is incurred [3].

Naturally fermented products based on protein-rich pulses are another example of products referred to or understood as meat substitutes. In soy-based tempeh, the mycelial growth of the mold Rhizopus oligosporus is used to lead the inoculated soybeans to the typical structure of the product. The structure of the product is derived from the random positioning of the substrate elements and their subsequent binding by the fungal mycelium. A characteristic feature of tempeh is that the growth of the mycelium takes place primarily in the spaces between the soybeans. A disadvantage compared to many other products is that the soybean used is uncrushed or in some cases partially crushed, but in terms of composition rather complete, whereby the products usually have a very typical “soy aroma” [4].

The firmness of the product results primarily from the comparatively high dry matter of the soybean, the texture is primarily due to the textural properties of the soybean or its fragments (“nibs”). The cavities in which the fungus can grow are defined by the random structure and shape of the soybeans and can hardly be influenced in the case of tempeh. Fermentation also requires a longer soaking phase of the soybeans. There are also references in the literature to okara-based tempeh, but here the entire mass is fermented without paying special attention to the structuring and combination of structural levels [5].

There is also a product known as tempeh, which is usually understood to be a fermentation with Rhizopus oligosporus, a mold that is typically used in Asia to ferment soaked soybeans. It is also known to use this term more broadly, where tempeh is understood to include the fermentation of grains or food processing by-products in addition to soybeans. A process is known in which the soybean is first cleaned, then boiled for 5-10 minutes, then soaked for 15-17 hours, then dehulled, washed and drained, after which inoculation with Rhizopus oligosporus is carried out, followed by fermentation for 35-37 hours to produce the finished tempeh. The first disadvantage with regard to the production process of the product is that the freedom of action with regard to the composition of the product is limited. In the mouth, the product appears compact and must therefore be processed through further process steps in order to achieve a mouthfeel similar to that of meat patties, for example. Another disadvantage is that the product is not very juicy. Another disadvantage is that due to superficial fermentation (the fungal mycelium only penetrates the outermost, superficial layer of the soybeans), the antinutritive substances present in the substrate can only be partially degraded enzymatically, so that the digestibility of the product cannot be favorably influenced [6].

### Task

The invention is based first on the task of creating a process for producing a product from one or more biological substances composed of substrates with differing dry matter contents, in particular a food/pharmaceutical/cosmetic product, which, compared to conventional products, is to be adjustable with regard to its sensory properties, such as texture and mouthfeel, depending on the area of application.

Furthermore, the invention is based on the task of providing a product manufactured by the process according to the invention, which can be used in a variety of ways, for example in the field of foodstuffs or cosmetics or in the pharmaceutical industry, the latter in particular for products medical in nature.

Finally, the invention is based on the task of using a product produced according to the process of the invention in a variety of applications, for example as a meat substitute or as textured elements in soups, curries, and other sauces or as a substitute for cream cheese products or as a matrix for absorbing and delivering active substances in the body or on the skin.

### Solution of the Task Concerning the Procedure

This task is achieved by a process for manufacturing a product from one or more biological substances or mixtures thereof, which, optionally after cleaning, after adjustment of the dry matter, optionally subsequent thermal treatment such as boiling, for example, as well as comminution and optionally further pre-processing to change the material properties and/or nutritional physiological properties of the starting material, are extruded, and through the extrusion process a strand or strands are arranged into a starting matrix that has channels, pores or cavities that are completely or partially open to the outside, and within which or between which one or more fungus/fungi and optionally additional fermenting microorganism(s) grow that before, during, or after the extrusion process are introduced or applied into or onto the starting matrix in the form of the vegetative or permanent form, and the said fungus/fungi cross-link with the starting matrix and/or grow in this while the said starting matrix is subject to a fermentation process or co-fermentation process, and the cross-linking and/or growth of the fungus/fungi decisively influences and/or partially influences the texture and/or firmness, and that the product produced from the starting matrix is if needed subsequently cut into pieces of predetermined dimensions, packaged, and supplied for further purposes of use whereby in the case of edible products, the taste and/or texture is determined by the fundus/fundi growing in the pores, channels and/or cavities and/or by other microorganism(s) introduced into the pores, channels, cavities and/or into the starting material(s) and/or by the duration and/or the temperature profile of the fermentation process and/or by the adjustment of the water content of the product during or after fermentation and/or by the composition of the biological starting material and/or by the volume fraction of pores, channels, cavities in the starting matrix and/or by the arrangement of the pores, channels, cavities and/or by the quantity of the interface between the totality of the strands and the totality of the pores, channels, cavities and/or by the diameter(s) of the strands and/or by gas exchange with the environment and/or by a process-technical pre-treatment adjusting the rheology of the starting material whereby the nozzle or nozzles and the support on which the starting material(s) is discharged from the nozzle or nozzles is/are movable relative to one another, so that either a chaotic distribution of the discharged matrix strands, forming a random heap, or a predetermined distribution of the discharged matrix strands in predetermined angular arrangements relative to one another is effected.

The first method is fast and inexpensive but does not permit a defined arrangement of the strands, so that structure/texture properties can be set within comparatively narrow limits. The second process is much slower but allows for a wide range in terms of strand arrangement and thus texturing. The second method is particularly advantageous if different textures and/or aroma perceptions are to be created in a spatially resolved manner within a product, i.e., if anisotropic distributions of starting material or several starting materials are necessary or desired. The first method is rather designed for random distributions of product strands/strand pieces and allows only very limited anisotropic structures.

The comminuted starting material, the substrate for fermentation, can be better interpenetrated by fungi and/or microorganisms than, for example, a soybean in tempeh fermentation, because it is de-structured due to the process. The depth of penetration can be controlled by the strand thickness. While in tempeh production the soybean or soybean pieces are the product-building element, in the new process these elements are created by extrusion of the starting material through one/multiple nozzles. The ratio of the volume of pores, channels, and cavities in the starting matrix to the volume of space occupied by strands and/or strand pieces is adjustable, as is the composition of the starting matrix or starting material (for example, nutritional physiological, optimized in terms of taste, or oriented towards the wishes of the consumer), which significantly influences the texture of the product. The interface necessary for fermentation and cross-linking of the strands with each other can be adjusted and thus so can the mechanical properties/texture of the product. Pre-processing, for example by an upstream extrusion process, can induce changes to the mechanical and/or rheological properties of the starting material, such as increasing the elasticity. In this case, the overall product texture may be strongly influenced by this pre-processing rather than by the superimposed fungal fermentation, whereby the contribution of fungal fermentation remains relevant, either for the texture characteristics and/or for the taste and/or the nutritional and physiological characteristics. While the fungal mycelial growth is desirable for, but not limited to, textural adjustment, co-fermentation with other microorganisms can modify the product in order to, but not only, alter the nutritional properties and taste. The fungi or their spores/permanent forms are either added to the starting materials to be extruded or applied to them after the extrusion process. The same applies to further microorganisms, which grow primarily in or on the starting matrix and may excrete extracellular compounds into unfilled spaces, while the fungi either grow into or on the starting matrix, or out of it, but also grow through the unfilled space between the strands of the starting matrix, thus creating a further structuring component for the product. The entire technological approach can in principle be applied to any conceivable starting material, provided that it allows at least one of the listed fungi to grow and can be arranged to form a matrix with channels, pores and/or cavities. A significant advantage of the process is also that only one starting material, such as okara, and one fungus or fungal spores is required to produce a meat-like structure/texture in a minimal embodiment, which is in marked contrast to other meat alternatives, that are usually composed of a variety of ingredients, including thickeners, stabilizers, and similar.

The texture and/or aroma can be variably adjusted by the interaction of different factors, whereby either different texture and/or sensory characteristics can be achieved with the same principle approach or different starting materials can be processed into products with similar sensory and/or texture characteristics.

A concentric layer containing at least 80% of the spores of the co-extrudate can be extruded by co-extrusion around a central strand piece, this layer representing 25 to 70%, preferably 40 to 60%, of the volume of the cross-section of the product strand or strand piece in relation to the co-extruded strand. Such a method is particularly advantageous, among other things, in order either to be able to reduce the required amount of inoculation material or, in the case of a fermentation with several fungi and/or microorganisms, to have them spatially separated from one another at the beginning of the fermentation.

### FURTHER INVENTIVE EMBODIMENTS

Patent claim 2 describes a process for the production of a structured body that is permeated with unfilled cavities (open channels, pores or cavities), solid, fermented, and is formed on the basis of modulable substrates (synonymous with starting material), wherein

(a) at least one rheologically and texturally adjustable, modulatable mass forms a directional or non-directional mesostructure (synonymous with starting matrix) that is freely adjustable in terms of arrangement over a wide range and that forms the substrate (synonymous with starting matrix) as well as the cavities (synonymous with pores, channels, cavities) for one or more fermentations and a basic structure (synonymous with starting matrix) that is co-decisive for the overall texture;
(b) by the introduction of at least one co- and/or superimposed microstructure (synonymous with fungal mycelium or network structure) induced by one or more fermentations, such that partially or completely interconnected filamentous network structures (synonymous with microstructure) caused by fungal growth are produced in, on and between the mesostructure elements (synonymous with extruded strands or strand pieces);
(c) by choosing the volume proportion of unfilled cavities, channels or pores, compartments in the total object, by choosing their arrangement as well as by choosing the ratio between mesostructure surface and mesostructure volume, the growth of the mycelium as a whole as well as the penetration and direction of the mesostructure with mycelium and thus in its entirety the network structures are adjustable and (d) the totality of the structuring elements at micro and meso level interact to effectuate an adjustable (i) solidification, (ii) rheological properties and (iii) sensory-relevant texturing.

According to patent claim 3, the process is characterized in that by the extrusion process simultaneous and/or in parallel and/or sequential extrusion process steps a body as a starting matrix is prepared, which consists of several extruded strands lying above and/or next to and/or behind one another, which are connected materially or functionally to connect to one unit via their mutually touching surfaces and form cavities, channels or pores between them, into which the fungus/fungi arrange/s itself/themselves. The pinpointed contact surfaces ensure that there is as large a surface area as possible provided for fermentation, through which cross-linking between the strands can take place. In addition, the network of cavities, channels and pores allows gaseous exchange with the environment so that, among other things, oxygen is also made available to the fungus/fungi. The arrangement of the strands and/or strand pieces is controllable by process engineering measures, i.e., the properties of the product, such as texture, can also be co-controlled via this method. The fungal mycelium growing in the cavities, channels and pores ensures the formation of one or an additional elastic product component and, depending on the product and its characteristics, a bite resistance and/or the perception of elastic components in the product mass during mastication.

According to patent claim 4, the growth and/or metabolic activity of the fungus/fungi can be interrupted and/or changed and/or controlled after a specific period of time for the respective starting material. Due to the extensive cavities, fluids can also be bound via capillary forces so that marinades, for example, can easily interpenetrate the product so that the adjustment of flavor is quick and easy.

Patent claim 5 describes a process in which the previously empty pores, channels or cavities occupied by the fungal mycelium/fungal mycelia after fermentation are partially or completely filled with a flowable and/or partially or completely solidifying material, wherein the solidification is carried out via an additional fermentation with the aid of a further bioactive organism, enzymatically, via thermo-reversible mechanisms, ionically induced, by heating or by other processes. The sensory perception or texture can be modified again by the filling, and it is also conceivable that the juiciness in particular is improved, which is a great advantage in the production of meat-like products, for example. In addition, aroma characteristics predestined for a first impression can be clearly emphasized if they can emerge more easily from the filling than from the fermented starting matrix. Furthermore, substances, flavors or materials can be added to the product that can change the growth of the fungus or fungi when present during the fermentation process. Therefore, the fermentation can thus also be indirectly controlled.

The extruded strands can have a ratio of nozzle diameter to diameter/equivalent diameter of the particles or structures contained in the mass of less than 1.5, preferably less than 2, especially less than 5, further preferably less than 10. During the extrusion process it should be ensured that the nozzles do not block. Depending on the material and the rheological and shape properties of the particles or structures, the lower critical ratio mentioned will vary.

The starting material for the starting matrix is processed by means of extrusion, co-extrusion or multi-extrusion processes to form a product strand and/or product strands and/or product strand pieces, the product strand subsequently remaining as a continuous strand or disintegrating into individual pieces and/or being disintegrated, and the temperature of the product strand and/or of the product strands and/or of the product strand pieces immediately at the die or perforated plate outlet being 2 to 99.5 [° C.], preferably from 5 to 99 [° C.], more preferably from 7 to 80 [° C.], more preferably from 10 to 70 [° C.], more preferably from 12 to 60 [° C.], more preferably from 12 to 45 [° C.], most preferably from 15 to 25 [° C.]—patent claim 6. The breaking up effect has the advantage that, in the case of a random structure/heap, the volume ratio between channels/cavities and extruded strands can be varied, as well as the average diameter of the channels/cavities. However, depending on the starting material, especially the selected dry mass, the extruded strand may already disintegrate automatically after exiting the extruder or may be random during defined extrusion due to rheological effects while the strand is being deposited. Co- or multi-extrusion processes offer the advantage that the rheological properties and functionality of the strands can be changed. For example, growth-promoting and growth-inhibiting starting materials can be combined in order to control the growth of fungi and/or microorganisms and thus the overall flavor formation as well as texture formation. Sensory problematic starting materials can also be hidden as an inner mass in a co-extruded strand, while the outer mass has a more accentuating effect. The same would apply to the visual design of the product. In the case of multi-extrusion, different starting materials could be combined in one product without having to mix them beforehand, which can have a beneficial effect on the texture of the product. Upstream heating with or without mechanical energy input can also be positive in order to adjust the—rheological properties such as elasticity process technically.

Patent claim 7 describes a process in which the extruded strand or strand pieces are foamed with gas inclusions caused by expansion of a compressed gas, e.g. CO₂, N₂O, O₂, or by gas formation in the course of fermentation, e.g., CO₂, by foaming of the material before discharge into the product, e.g., with CO₂, O₂, N₂, air, or by a chemical reaction, e.g., that of a carbonate with an acid, or by the expansion of water to water vapor within the strands or strand pieces. This allows a caloric reduction, also a change in texture, and depending on the gas, also promotion of an internal fermentation, which may change the sensory properties as well as the texture.

The oxygen required during fermentation is supplied to the fermenting matrix. Since most fungi/molds require oxygen for growth, the addition of oxygen can promote growth.

The biological substances for the starting matrix are subjected to a thermal or other treatment such as PEF or high pressure and the total bacterial count, based on the initial bacterial content, is reduced by 50 [%], preferably by 90 [%], further preferably 99 [%] or 99.9 [%] or 99.99 [%] or 99.999 [%]. This results in a reduction of wild fermentations, for example associated with the development of off-flavors. In addition, the risk of the growth of any pathogenic microorganisms can be reduced. A swelling of the material in preparation for extrusion improves extrudability.

Advantageously, according to patent claim 8, the fungi/fungus spores/molds/mold spores used for the fermentation originate from the genus Rhizopus, for example Rhizopus oligosporus, Rhizopus stolonifer, Rhizopus oryzae, Rhizopus arrhizus and/or from the genus Actinomocur, for example Actinomocur elegans spp. meitauza and/or from the genus Aspergillus, for example Aspergillus oryzae and/or from the genus Penicillium, for example Penicillium candidum, Penicillium camemberti, Penicillium roqueforti, Penicillium glaucum, and/or from the genus Geotrichum, for example Geotrichum candidum, and/or of another genus capable of modifying the texture and/or sensory characteristics of the product, as well as the microorganism(s) used for the microbial fermentation or co-fermentation from the genus Bacillus, for example Bacillus subtilis spp. natto and/or from the genus Neurospora, for example Neurospora intermedia and/or from the genus Lactobacillus, for example Lactobacillus bulgaricus, Lactobacillus reuteri and/or from the genus Lactococcus, for example Lactococcus lactis and/or from the genus Propionibacterium, for example Propionibacterium freudenreichhii and/or from the genus Zymomonas, for example Zymomonas mobilis and/or from the genus Leuconostoc, for example Leuconostoc mesenteroides and/or from another genus which is suitable for changing the texture and/or sensory properties of the product. Depending on the microorganism(s), a different sensory and texture outcome of the product is obtained.

According to claim 9, the inoculation of the starting matrix with fungal mycelium and/or fungal spores and/or mold mycelium and/or mold spores is carried out in such a way that they are, for example, mixed with the starting material and/or sprayed onto the starting matrix and/or the product is soaked in and/or with a suspension of said fungal mycelium and/or said fungal spores and/or said mold mycelium and/or said mold spores. The formation of a fungal mycelium from comminuted pieces of mycelium or fungal spores ensures cross-linking of the starting matrix. The different variants take into account the fact that in some extrusion techniques the inoculum must be added to the product later, as it would not survive the extrusion process undamaged, for example when higher temperatures are applied.

After fermentation, the fermentation products are de-structured, whereby the products formed from the fermented starting matrix during fermentation are divided into smaller units by crushing, chopping, or dividing. The shredded material can serve as a pre-structured starting material for further products, which are structured in a superordinate manner and assembled together in a new way, as well as cross-linked with each other. Side streams from food production are used with largely insoluble components such as natural fibers and non-water-soluble proteins to prepare the starting matrix.

The extruded strands form a starting matrix with different diameters of the different strands in a cross-section orthogonal to its longitudinal axis. The strands are arranged on top of each other in the form of a network and form channels, pores, or cavities between them.

In the method according to claim 10, the starting material is conveyed in the extrusion process through nozzles or openings, such as in a perforated plate, with an inside diameter of 0.4 to 9 [millimeters], preferably 0.5 to 7 [millimeters], preferably 0.8 to 5 [millimeters], preferably 1 to 3.5 [millimeters], again preferably between 1 and 2.5 [millimeters], in particular 1.1 to 2 [millimeters], the diameters of the openings having the same or different diameters in the case of parallel or consecutive extrusion processes. Due to different diameters of the strands and, as a consequence, due to different relative penetration depths of the fungi, the mechanical properties of the fermented products can be significantly changed. Due to the different structural resolutions, an altered de-structuring behavior in the mouth also induces different textures. Also suitable for changing the texture are product strands or strand pieces of different thickness in the same product in the case of the chaotic heap, as the combination of the mentioned different relative penetration depths in connection with different sized channels, cavities, pores changes the texture.

The strands forming the starting matrix to be fermented are produced by extruding the starting material through perforated plates with openings of 0.4 to 9 [millimeters], preferably 0.5 to 7 [millimeters], preferably 0.8 to 5 [millimeters], preferably 1 to 3.5 [millimeters], further preferably 1 to 2.5 [millimeters], in particular 1.1 to 2 [millimeters], the diameters of the openings having the same diameter or different diameters. Perforated plate extrusion allows a large number of strands to be extruded in parallel, so that the production speed is greatly increased. Different diameters can lead to an increase in the packing density in the heap and the cross-linking can be adjusted by fermentation, with effects on sensory properties and texture. The openings in the perforated plate have different dimensions.

The heap of statistically randomly arranged strands and/or strand pieces is shaped and/or compacted after the extrusion process, whereby the strands or strand pieces are partially pressed together and connect materially or functionally into one unit via the surfaces. This results in an improved production speed combined with the shaping into a product. The creation of the starting matrix for fermentation and the forming process are separated from each other. The internal structure of the starting matrix can be changed by pre-compaction, with effects on the cross-linking during fermentation and on the texture as a whole.

In the case of a co-extruded starting matrix to be fermented, the mass proportions are changed relative to each other at least once on the entire extruded strand during the extrusion process. This would allow locally different starting material ratios to be achieved in one strand with a single extrusion process.

By co-extrusion, a concentric layer can be coated around a strand/strand piece, in which at least 80% of the spores of the co-extrudate are contained, whereby this layer, in relation to the co-extruded strand, accounts for 25 to 75%, preferably 40 to 60%, of the cross-section of the strand/strand piece.

Fermentation is carried out at a relative ambient humidity of between 40 and 100%, preferably between 50 and 99%, more preferably between 60 and 99%, again preferably between 70 and 98%, in particular between 75 and 95%, relative to the atmosphere surrounding the product.

The substrate phase is produced by directional or non-directional 3D extrusion.

The dimensions of the body correspond in all spatial directions to at least three times the characteristic diameter of the mesostructural elements, preferably at least five times, in a more preferred embodiment ten times and in an even more preferred embodiment twenty times the characteristic diameter of the mesostructural elements.

The phases used to create the mesostructure are pasty, extrudable masses with yield stress, for example based on plant-based, protein-containing, fiber-containing products, for example okara, marc, pomace, etc. After leaving the extrusion device, the strand shape is largely retained and does not flow unless a yield stress is given, which must usually be the case.

The biological substances or mixtures thereof, optionally also with additions of further substances, comprise substances that allow and/or promote a desired germination and/or growth and/or metabolic activity of the fungus/fungi and/or their spores/permanent forms and optionally of the microorganisms) and/or their spores/permanent forms due to the material composition as well as the set dry matter content and/or further suitable treatment steps, for example, biological substances, or mixtures thereof, with increased protein content in the dry matter, such as, for example, peas, soy, quinoa, chickpeas, tofu, seitan, gluten, cream cheese masses, processed cheese masses, ricotta, and/or with increased fiber content in the dry matter, such as okara, spent grains, whole grain cereal products, lamely insoluble residues from fat/protein extraction and/or increased fat content in the dry matter, such as almonds, cashew, soy, and/or high carbohydrate content in the dry matter, such as wheat or other cereals or pseudo-cereals and/or hydrocolloid-based such as gels based on gelatin, pectin, starch, optionally with further additives and/or paste-based, such as pastes based on concentrated dispersions of any powders or powder mixtures, such as milk protein, whey protein isolate or plant-based protein concentrates or isolates, optionally with further additives and/or already fermented, subsequently re-comminuted material, wherein in each case the adjustment of the water content is effected in such a way that the substances have a yield stress or that the yield stress is thermally induced, for example in the form of thermo-reversible gelation. In principle, all materials/substances are conceivable that provide as a minimum the necessary growth conditions for the fungus used that it may propagate and thus form a mycelium. The dry masses can be highly variable; in the case of hydrocolloids, gel-like structures can already be formed with a dry matter of less than one percent by mass; in the case of some very oily seeds, dry matter of even more than 60 percent by weight can be usefully extruded to form a starting matrix. The starting materials can be, for example, intact biological substances such as seeds, but also intermediate products of a process such as, for example, the still malleable tofu mass after precipitation or a processed cheese mass, as a starting material originating from a finished product.

The growth and/or the metabolic activity of the fungus/fungi growing in the pores, channels or cavities of the starting matrix and/or of the microorganism(s) growing or metabolically active in, on or between the strand(s) is controlled and/or partially or completely ended thermally and/or by gassing with, for example, CO₂, N₂ or mixtures thereof and/or by changing the fermentation conditions such as the relative humidity and/or temperature and/or by filling the pores, channels, cavities and/or by a high-pressure treatment and/or by cooling and/or by freezing and/or by other suitable methods, during or after the fermentation. In this way, improved or stable or largely stable textures or an improved or stable or largely stable aroma/flavor profile can be achieved by such measures resulting in further changes can be slowed down, adapted, or completely eliminated. The formation of desired flavors and/or textures can thus be induced or the development of undesired flavors and/or texture changes can be slowed down or completely eliminated.

The water content of the starting matrix is changed during or after the fermentation process. The texture properties of the product can be controlled or co-controlled during or after the fermentation, as can the growth and/or metabolic activity of the fungi and/or microorganisms. The fermentation and/or downstream modifying processes can also be controlled.

The biological starting material or materials is or are subsequently cut to predetermined size(s) during the extrusion process in the form of a continuous strand. Compared to a locally predetermined deposition of the strands, a random arrangement of strand pieces produced as described above can lead to specific textures, with the advantage of a higher production speed and reduced production costs. By choosing the average length of the strand pieces, the average diameter of the pores, channels, and cavities can be controlled, in turn changing the texture of the product. By varying the length and/or thickness of the strands, the degree of packing of the starting matrix can also be adjusted.

The pores, channels or cavities not filled by the fungus/fungi are completely or partially filled with flavorings and/or vitamins and/or antioxidants during or after the fermentation process.

The pores, channels or cavities not filled by the fundus/fundi are filled or supplied completely or partially with medicines and/or wound treatment product, for example ointments, antibiotics, burn ointments and/or similar, during or after the fermentation process.

The starting matrix is prepared from the starting material okara as follows:
Step 1: okara with a dry content of 15 to 25 [% by weight] is used as raw material, as it is produced in soy milk and tofu production;
Step 2: the okara is heated to 95+/−1 [° C.] with constant stirring and held there for 60+/−1 [minutes]. The mass is then stirred further and cooled to 40+/−1 [° C.];
Step 3: the pH of the okara mass is adjusted to 5.2+/−0.1 by the addition of lactic acid (80 [%, by weight]);
Step 4: the treated okara is pressed through a filter cloth with a mesh size of 0.5 [millimeters] to obtain a dry content of 25+/−0.5 [%, by weight];
Step 5: the mass is transferred to pacojet containers and frozen at −22 to −25 [° C.]; the frozen mass is comminuted with the Pacojet PJ2E (Pacojet AG, Zug, Switzerland) using the “standard” pacotizing blade and splash guard with pre-scraper; the particle size is reduced to a D₉₀ of 600 to 800 [microns], particle size measurement is carried out in a Beckmann Coulter Counter LS 13320, with water modulus at 20+/−1 [° C.];
Step 6: per 1500+/−10 [g] okara mass, add 10+/−0.1 [g] Rhizopus oligosporus starter culture (Makrobiotik Hohrenk, Germany);
Step 7: the mixture is mixed in a Kenwood Major Swiss Edition mixer for 5 [minutes] on speed 5, and then transferred to a sterile plastic bag with a layer thickness of 25 [mm], vacuumed to a pressure of 200 [mBar] and brought to a temperature of 20+/−1 [° C.];
Step 8: the mass is transferred into tubular extrusion cartridges through a cut open corner of the plastic bag, as free of air as possible;
Step 9: the transferred okara mass (also called substrate) is kept at 20+/−1 [° C.] and is ready for extrusion;
Step 10: the mass is then extruded through a 1.8 [millimeter] nozzle, building up a CAD-defined object layer by layer on a glass, steel or plastic plate; the process is analogous to the fused deposition modelling process in 3D printing, building up two-dimensional layers on top of each other to generate three-dimensional objects; this is done at an ambient temperature of 20+/−1 [° C.] and a humidity of 85 [%];
Step 11: the generated objects are transferred to an incubator (Binder APT.line™, with microprocessor program RD3, Binder GmbH, Germany) and fermented for 48+/−2 [hours] at 25+/−1 [° C.] and 85 [%] humidity. The objects are covered with parchment paper (type irrelevant) during fermentation;
Step 12: after fermentation, the objects are transferred into sterile plastic bags and vacuumed at 200 [mBar];
Step 13: the filled and vacuumed bags are blast frozen to −17 to −19 [° C.], and stored at this temperature until use.

Rhizopus oligosporus is considered a safe organism on a soybean substrate, so that no approval problems are to be expected in Europe in the sense of the Novel Food Regulation. There is currently no efficient use for okara, so that an estimated 3 million tons of okara are fed to animals or biogasified worldwide every year. Okara is considered to be nutritionally beneficial due to its high fiber content.

The extruded strands can be cut into predetermined sizes in a downstream process, for example by a rotating knife, after the strand exits the extruder.

The fermentation process of the product is carried out at temperatures between 10 and 50 [° C.], preferably between 12 and 45 [° C.], further preferably between 15 and 35 [° C.], further preferably between 15 and 32 [° C.], in particular between 18 and 28 [° C.] and in some fermentations the temperature is changed during fermentation. The growth of the microorganisms as well as their metabolism can be controlled by the fermentation temperature. In a fermentation consisting of more than one organism, the relative growth of one compared to another and the temporal dominance of one organism can be controlled, effecting upon sensory properties and texture.

The fermentation is carried out at a relative ambient humidity between 30 and 100 [%], preferably between 30 and 98 [%], in particular between 40 and 95 [%], further preferably between 55 and 95 [%], for example in particular between 70 and 95 [%], relative to the atmosphere surrounding the product. By changing the dry matter during fermentation, the growth of the fundus/fundi and/or microorganism(s) as well as the structure of the product is advantageously changed. For example, depending on the starting material, the product will become less soft or remain firmer to the bite if the water content is reduced during the cooking process.

The air flow velocity of the atmosphere surrounding the product is less than 50 [cm/s], preferably less than 15 [cm/s], more preferably less than 5 [cm/s], even more preferably less than 1 [cm/s], in particular less than 0.5 [cm/s], for example less than 0.1 [cm/s]. Especially in the case of Rhizopus oligosporus, an excessive flow of air/gas must be avoided, otherwise the mycelium will start to sporulate (grey/black coloration of the product on the surface). On the other hand, excessive air flow can regulate water removal. As a compromise, the starting matrix can also be packed in perforated bags for fermentation, similar to what happens in tempeh production, but this is at the expense of water exchange with the environment.

The dry matter of the product at the start of the fermentation is preferably 0.5 to 70 [weight percent], in a more preferred embodiment 1 to 60 [weight percent], in a still more preferred embodiment 1.5 to 55 [weight percent], in a still more preferred embodiment 2 to 50 [weight percent], in a still more preferred embodiment 3 to 50 [weight percent], in a still more preferred embodiment 5 to 45 [weight percent], in a still more preferred embodiment 7 to 40 [weight percent], most preferably 15 to 40 [weight percent]. Depending on the desired product, the dry matter can be chosen very differently and also depends very much on the material used, in particular on the amount of low-molecular constituents as well as the fat content.

By adjusting the ratio of unfilled and filled spaces, the overall texture can be adjusted by changing the fungal growth as well as the overall mechanical properties of the product.

The masses used are at least one or more different masses, each of which is composed of one or more, preferably co-extruded, phases, whereby the composition can be changed dynamically during the application process. By combining several starting materials without mixing, it is possible to directly influence the texture of the product. By co-extruding different masses, the mechanical properties and the penetration of the strand with mycelium can be adjusted with corresponding effects on the overall texture. The overall sensory properties of the product are also adjusted.

The body is permeated by filament-like network structures caused by fungal growth. Elastic components are introduced by the fungal mycelium with regards to the mechanical properties of the product. This is based on extensive cross-linking of the mycelium with each other and with the substrate on which the fungus grows.

The fermentation leading to the formation of partially or completely coherent, filamentous network-structures caused by fungal growth is carried out with one or more fungal cultures and comprises a volume fraction of the volume of the unfilled cavities of at least 0.1 [%].

The spores forming the mycelium are isotropically distributed in a mass of a typical cross-section of the mesostructural elements formed therefrom, and/or are concentrated in the outer 40% (v/w) of the mesostructural elements, and/or is the distribution of spores in a typical cross-section of the mesostructural elements exhibiting a gradient from the center towards the location of maximum distance from the center, or are found at least 95% on the surface of the mesostructural elements.

Within a product, spores of different genera of mycelium-forming filamentous fungi, for example Rhizopus oligosporus, Actinomucor elegans and, if necessary, additional microorganisms such as Propionibacterium freudenreichhii, Zymomonas mobilis, whose preferred spatial localization in the body can be described as isotropic or defined anisotropic, are used. The spatially different distribution of the fermentation organisms can produce locally different textures, which is expressed in an altered superordinate texture perception. In addition, it is conceivable that a co-fermentation is carried out with microorganisms that behave differently next to each other than they do separately with regard to the overall aroma.

When using several different genera of spore-forming fungi (especially molds), these are preferably present in the same or different compartments before fermentation begins.

### Solution of the Task Concerning the Product

Patent claim 11 is characterized in that, in the case of edible products, the taste and/or texture is determined by the fungus (i) introduced into the pores, channels and/or cavities and/or by further microorganisms introduced into the pores, channels, cavities and/or into the starting material(s) and/or by the duration and temperature profile of the fermentation process and/or by the adjustment of the water content of the product during or after fermentation and/or by the composition of the biological starting material and/or by the volume fraction of pores, channels, cavities in the starting matrix and/or by the arrangement of the pores, channels, cavities and/or by the quantity of the interface between strands and pores, channels and cavities and/or by the diameter(s) of the strands and/or by gas exchange with the environment and/or by a technical pre-treatment process adjusting the rheology of the starting material. The texture and/or aroma can be pronounced differently due to the interaction of different factors, whereby either different texture and/or sensory characteristics can be achieved with the same principle approach or different starting materials can be processed into products with similar sensory and/or textural characteristics.

The product consists of at least one layer, preferably two or more layers, of extruded strands or strand pieces, integrally connected to each other, materially or functionally, of one or more biological material(s) and one or more fungus/fungi/mold(s) and optionally microorganism(s), wherein between adjacent extruded strands of the starting matrix there are wholly or partially, outwardly open cavities, pores or channels that are wholly or partially filled by the relevant fungus/fungi/mycelium and/or microorganism(s) or by its/their excretory products. By spreading the starting material via extrusion processes, (a) the contact area for fermentation can be defined, (b) the space for fungal mycelium growth and/or microorganism growth and/or for the accumulation of segregated products and thus the de-structuring behavior in the mouth, the flavor perception, as well as the mechanical product properties can be adjusted. The largely interconnected cavities, pores and channels with connection to the product surface allow for an oxygen-requiring fermentation. It is also conceivable to create several independent, not directly connected initial matrices, which are connected to each other via fermentation, such as several nested high cylinders of different diameters, which do not touch each other, but are grown together/connected with fungal mycelium in the course of fermentation. The partial filling of the spaces with mycelium allows the product to be subsequently equipped with another phase that can have aromas or other sensory-relevant functions.

Patent claim 12 describes a product in which several layers or sheets of extruded product strands are arranged in predetermined or chaotic angular arrangements above and/or next to and/or behind each other. This gives the advantage of different textures with different speed and positioning accuracy of the strands.

The product according to patent claim 13 is characterized in that the starting material for the starting matrix consists of biological substances or mixtures thereof that allow and/or promote a desired germination and/or growth of the fungus/fungi or their spores/permanent forms and optionally of the microorganism(s) or their—permanent forms due to the material composition as well as the adjusted dry matter content and/or further suitable treatment steps, such as, for example, biological substances with increased protein content in the dry matter, such as, for example, peas, soy, quinoa, chickpeas, tofu, seitan, cream cheese masses, processed cheese masses, ricotta and/or with increased fiber content in the dry matter, such as okara, spent grains, whole grain cereal products, largely insoluble residues from fat/protein extraction and/or increased fat content in the dry matter, such as almonds, cashew, soy and/or high carbohydrate content in the dry matter, such as wheat or other cereals or pseudo-cereals and/or hydrocolloids such as gels based on gelatin, pectin, starch and/or pastes such as concentrated dispersions of any powders such as milk protein, whey protein isolate or plant-based protein concentrates or isolates, whereby in each case the adjustment of the water content is effected in such a way that the substances have a yield stress. Different starting materials can be used for this product, so that the overall product can also be nutritionally and/or physiologically optimized by mixing relevant masses or any other ingredients.

The channels, pores or cavities are only partially filled by the fungus/fungi and/or microorganism(s) or their excretory products and in the remaining cavities or the like another substance, for example flavor enhancers and/or vitamins and/or antioxidants and/or colorings and/or flavorings, is arranged. The structure of the product allows different substances to be subsequently added to the product depending on the requirements so that the same matrix can be used to produce differently flavored products, for example.

The cavities, channels or pores are covered with an anti-inflammatory and/or healing medication.

The cavities, channels or pores that are not filled by the fungus are covered with a cosmetically active substance, for example a cream, an anti-ageing agent or the like.

Advantageously, the product according to patent claim 14 is characterized in that the proportion of cavities, channels or pores is 20 to 85 [%, volume fraction], preferably 20 to 75 [%, volume fraction], again preferably from 25 to 75 [%, volume fraction], again preferably between 25 and 70 [%, volume fraction], in particular preferably between 25 and 60 [%, volume fraction], most preferably between 30 and 55 [%, volume fraction]. The volume fraction of pores, channels and cavities (and the distribution of these) essentially determines the overall texture, as this can be used to control the growth of the mycelium and its overall mechanical properties.

Claim 15 is characterized in that the firmness of the product measured after fermentation compared to the firmness of the underlying starting matrix before fermentation increases by at least a factor of 20, preferably by at least a factor of 12, more preferably by at least a factor of 8, even more preferably by at least a factor of 5, even more preferably by at least a factor of 3.5, even more preferably by at least a factor of 2, even more preferably by at least a factor of 1.5, more preferably by at least a factor of 1.2, most preferably by at least a factor of 1.1, wherein the firmness is determined as the maximum force with a penetration measurement by means of a flat, round cylinder geometry with a diameter of 8 millimeters, which penetrates at a speed of 0.5 cm per second into a product body of the dimension 20 millimeters×20 millimeters×20 millimeters with a penetration depth of 10 millimeters at room-temperature.

### Solution of the Task Concerning the Use

The use according to patent claim 16 is characterized in that the product is usable as a meat substitute.

The product can be used as a bandage or dressing pad for wound treatment. The product can also be used in a variety of cosmetic applications, for example as a face mask and contains skin-caring substances.

A product according to the invention is usable as a meat-like patty.

The product is used as a spreadable, textured mass, for example as a cream cheese or spread.

The product can serve as lasagna sheets, noodles, or other pasta-like products.

The product can be used as a flavoring powder after grinding in soups, in sauces or as a seasoning.

The mass used has a lipid content of 0 to 70 [weight percent], a protein content measured as nitrogen of 0 to 95 [weight percent], a fiber content of 0 to 80 [weight percent] and a carbohydrate content of 0 to 95 [weight percent], as well as other ingredients, in each case based on the dry mass.

The masses are composed of, or spatially structured in such a way that, due to their composition, they inhibit or favor the growth of the organisms forming the interconnected, filament-like network structures caused by fungal growth.

For the formulation of the phases, soluble proteins, in the sense of the Osborne classification albumins and globulins, and water-insoluble proteins, in the sense of the Osborne classification prolamins and glutelins, are constituents, these proteins being present in all purification grades from a protein content of more than 0.01%, in a preferred embodiment of more than 0.1% in a still more preferred embodiment of more than 1% and in a still more preferred embodiment of more than 5%, measured in the substrate before fermentation as total nitrogen, in a preferred embodiment as oligopeptides or larger, in a still more preferred embodiment as polypeptides or larger, in each case alone or in mixtures as the gravimetrically predominant element.

The combination of mesostructuring by a mass having a yield stress and one or more fermentations for microstructuring is also proposed. The mesostructure serves as (a) a fermentation scaffold and substrate for fungal and/or other fermentation and (b) a phase that contributes to the overall texture and sensory characteristics. The extent and structure of fungal growth is controlled by arranging the substrate of the fermentation organism(s), for example a fiber-rich, plant-based mass into a 3D structure and 3D shape, each in the x,y,z direction, for example by a strand-like 3D microextrusion in space. By providing unfilled cavities in the structure, tailor-made growth conditions (of a tailor-made nutrient and/or oxygen supply and/or moisture distribution and/or retention) of the inserted fungus (i) and/or other microorganism(s) and thus a defined and directed growth of the fungal mycelium between and into the substrate are adjustable according to the invention.

Preferably, the dimensions of the body in all spatial directions correspond to at least three times the characteristic diameter of the mesostructural elements, preferably five times, in an even more preferred embodiment ten times and in an even more preferred embodiment twenty times the characteristic diameter of the mesostructural elements.

The dry matter of the product is preferably 1-50% (w/w), in a more preferred embodiment 3-45% (w/w), in an even more preferred embodiment 5-40% (w/w), in an even more preferred embodiment 7-35% (w/w).

The mesostructure elements used each comprise at least one or more different masses, which may each be composed of several, preferably co-extruded, phases, the composition being dynamically variable during the dispensing process.

The body is advantageously permeated three-dimensionally by filamentous network structures caused by fungal growth, whereby the partially or completely interconnected filamentous network structures caused by fungal growth are formed by fermentation with one or more fungal cultures and comprise a volume fraction of the volume of the unfilled cavities of at least 0.1%.

Advantageously, (i) the spores forming the mycelium are isotropically distributed in a mass and in the typical cross-section of the mesostructural elements formed therefrom, and/or (ii) are predominantly concentrated in the outer 40% (v/w) of the mesostructural elements, and/or (iii) and/or is the distribution of spores in a typical cross-section of the mesostructural elements exhibiting a gradient from the center towards the location of maximum distance from the center, or (iv) at least 95% is found on the surface of the mesostructural elements.

Within a product, spores of different genera of mycelium-forming filamentous fungi, for example Rhizopus oligosporus, Actinomucor elegans or microorganisms such as Propionibacterium freudenreichhii, Zymomonas mobilis, whose preferred spatial localization in the body can be described as isotropic or defined anisotropic, can be used.

When using several different genera of spores, they are preferably present in the same or different compartments before fermentation begins.

At least one mass has a lipid content of 0-70% (w/w), a protein content measured as nitrogen of 0-50% (w/w) and a carbohydrate content of 0-80% (w/w).

The mass may further be composed or spatially structured in such a way that, due to its composition, it inhibits or favors, overall or spatially locally, the growth of the organisms forming the interconnected, filamentous network structures caused by fungal growth.

This adjustability of growth is achieved, for example, by the ratio of substrate volume to the volume of unfilled cavities, by the ratio of surface area to substrate volume, by the overall structure of the substrate, the absolute diameters of the substrate strands, by the substrate composition and substrate dry matter, as well as the spatial arrangement of substrate and empty/unfilled cavities, and the suitability of the structure to be able to carry out any kind of gas exchange with the atmosphere surrounding the object/body (forced or unforced, directly or mediated via interrelated mycelial structures).

The purposefully created substrate-free cavities are preferably interconnected and basically allow the exchange of gas with the atmosphere surrounding the object, so that oxygen necessary for fermentation can migrate into the product, but also the gas atmosphere can be set within the product. The growth of a microscopically and/or mesoscopically and/or macroscopically coherent and/or interpenetrating fungal mycelium solidifies the substrate, the individual substrate elements/strands are bonded to each other and the overall object is significantly stronger and more elastic from a rheological point of view. Compared to the production of tempeh, the process according to the invention offers great freedom with regard to the selection and composition of the substrate, for example with regard to sensory, growth—modulation by promoting and inhibiting substances, the substrate properties, as well as the directed and specifically adjustable formation of the overall structure and overall firmness and texture through the combination of three-dimensional substrate arrangement and superimposed fungal fermentation. Another advantage over the classic soybean-based tempeh process is that insoluble proteins as well as fibers and other ingredients derived from the natural matrix can be used in any mixture, preferably directly from the moist, non-dried side stream of a conventional production process. For example, okara, a side stream of soy milk and tofu production, can be used as a substrate. This means that very inexpensive substrates can be used, which can also be optimized by mixing with other masses. The combination of 3D substrate arrangement with fungal fermentation leads to a new class of structured objects/products that can be further developed as a meat alternative, among other things.

As an alternative to the 3D substrate arrangement by extrusion in the x,y,z-direction, it is possible for the production of higher throughputs to extrude one or more substrate phase(s), for example via a perforated plate, in an x,y-direction into, theoretically, infinitely long parallel strands of any diameter, diameter distribution and, to a limited extent, also in the z-direction by rotating or periodically oscillating feeds in extrusion direction and thus to orientate them. Optionally, the parallel strands can also be arranged randomly to form an object and such an object can be shaped and/or compacted by further suitable measures.

The structure of the applied substrate mass has repetitive elements, especially in the case of small product units, but there may also be macroscopically anisotropic structural elements. The masses are applied, for example, by extrusion strand by strand, but can also be achieved by other methods with comparable results.

In a basic embodiment, the substrate phase may be interpreted as an emulsion, suspension or suspoemulsion. In one possible embodiment, the substrate phase is dispensed as a foam. The overrun is between 1 and 200% and is preferably generated by expansion of a dissolved gas or, for example, by gas release from chemical substances, but in a further embodiment also by water evaporation in conjunction with a preceding pressure phase.

The three-dimensional structure of the substrate mass, in turn, indirectly determines the overall structure formed by the fungal fermentation and which directs the internal cross-linking by fungal mycelium. In contrast to the classic tempeh fermentation with soybeans, the resulting product structure and texture can be controlled. Through the special arrangement of the substrate strands in combination with the adjusted rheological properties of the substrate mass, different structure and texture perceptions can be created, some of which can be described as meat-like.

Suitable substrates are various masses composed of proteins (0-100%) and/or carbohydrates (0-100%) and/or fats (0-50%) and other minor components (<10%), which are such that they are extrudable. For example, extrudable from dies in a range of 0.1-30 mm, where the compounds are matched to the dies. Preferably, masses are used that represent side streams from conventional production, such as okara, cereal bran, press cakes from oil extraction as well as mixtures thereof, fruit and vegetable pomace, brewer's grains, sugar beet press residues. Nutritional physiological or sensory deficits of these masses are preferably corrected by mixing them with other masses. Typical dry matter content of such masses are 15 to 85% (w/w); these dry matters can be adjusted by mechanical dehydration, but also by partial thermal processes or combinations. To increase the availability of nutrients for the fungal mycelium or other (partial) fermentations and to modify the flow properties and/or particle size distribution of the masses, the masses can be further mechanically digested (for example, by fine grinding, ball milling or cryomechanical-abrasive processes such as processing in the Pacojet). For hygienic and technological reasons, the masses may be subjected to a step that reduces the microbial count, such as thermal treatment or applications of PEF, high pressure, US or combinations thereof, prior to their use as a substrate. A further effect of such a treatment can be the release, and associated with that an easier utilization, of the substrate contents by the fungal mycelium or the formation or degradation or removal of sensory relevant compounds or precursors thereof, as well as compounds that lead to a desired adjustment of the sensory properties of the product through fermentative activity.

The substrate phases can be additionally modified by incorporating further materials/substances such as carbohydrates, hydrocolloids, cross-linking ions. In a further embodiment, fermenting microorganisms and/or fungi and/or enzymes can be added, for example, which can lead to cross-linking and/or solidification and/or modification of the rheological properties of the substrate phase before, during or after fermentation.

The basic phases used to produce the mesostructure are pasty, extrudable masses with yield stress, based on plant-based fiber-containing products, for example okara, marc, pomace, etc., based on protein-rich products such as tofu masses, gluten or other protein-free masses, etc., whose proteins can be soluble or insoluble and are present in different concentrations, dried or as a flowable concentrate or isolate.

Oxygen is supplied to the object through a largely continuous network of unfilled cavities. The corresponding volume share of unfilled cavities is between 10% and 80% and is determined by the arrangement of the substrate network. The unfilled cavities are traversed and filled by fungal mycelium depending on the distance between the limiting substrate strands and the growth conditions. The growth can be controlled, so that on the one hand the penetration of the substrate, as well as the connection of the substrate strands and thus the overall product properties can be defined. The growth can also be modulated by the partial oxygen pressure and the absolute amount of oxygen available. Within an object, different growth conditions can be set by locally different ratios between the substrate phase and the gas phase in order to be able to generate macrostructures in a targeted manner.

One advantage of the free choice of substrate is that two or more different substrate phases can be used without further ado. A major advantage of this option is that the micro-, meso- and macrostructure can be changed in a targeted manner, which has an effect on texture, sensory and optics. In a simple embodiment, different concentrated phases of the same material can be used. In a more complex embodiment, different materials can also be used. For example, tofu mass and okara mass can be processed into a joint product, whereby the separate phases are simultaneously used as structuring means, as they can have different rheological and textural properties due to their composition, and can also be penetrated to different degrees by the fungal mycelium, which can also lead to rheological and textural differences.

In order to adjust texture and/or rheological characteristics, growth-promoting and growth-inhibiting (or rather growth-favorable or rather growth-unfavorable) substrate phases can be used specifically with regard to penetration or cross-linking by fungal mycelium. The different phases can either be produced by separating a common starting material or can also comprise completely different starting materials. In addition to the use of antinutritive factors, an increase in the fat content of the substrate used can also be considered as a growth inhibitor. In addition to the dispersed presence of fat, fat can also be used as a coating of a substrate strand completely or partially in order to impede or prevent growth into the substrate phase at the coated/covered sites. Instead of placing a growth-promoting phase and a growth-inhibiting phase in relation to each other, these can also be co-extruded like the described fat phase, so that the growth of the fungal mycelium into the two-phase substrate strand can be limited, provided that the growth-inhibiting phase is found in the core of the strand. This may be desirable to adjust texture or generally limit the amount of fungal mycelium without sacrificing essential texture adjusting or rheological features.

In one possible embodiment of the invention, all compartments of the object that are not filled with substrate phase or fungal mycelium are filled with a further, flowable phase after a (first) fermentation and solidified to different degrees. The solidification can take place (i) via a continued fungal fermentation, (ii) an additional fermentation with the aid of a further bioactive organism, (iii) enzymatically, (iv) via thermo-reversible mechanisms, (v) ionically induced or other processes and can be different in the interior of the object compared to areas near the surface (e.g.: final, solidified second phase, still flowable in the interior). The flowable phase can have different compositions, different dry masses and different rheological properties. Preferably, the same material is used that already makes up the continuous substrate phase, also preferably material that has been partially or completely separated from the material used as substrate phase in one or more preceding steps, with or without mixing with other materials, in particular those that allow or make possible an adjustment of the firmness or the rheological properties in the gelled/solidified state. In particular, fat and/or hydrocolloids/carbohydrates, emulsified or dispersed, can be added to modulate the sensory properties. The filling of the compartments can take place at different times during the fermentation of the substrate phase and with different characteristics of the fungal mycelium in order to be able to use a solidifying activity of the fungal mycelium if necessary. In a further embodiment, the filling can also take place at any later time, for example before further processing or by a consumer. In a further embodiment, substances can be added to the filling phase that subsequently change the properties or growth of the fungal mycelium or the substrate phase.

In an exemplary embodiment, okara with an initial dry matter of 18% is adjusted to a dry matter of 21% by mechanical pressing, mixed and homogenized with soybean oil (5%, w/w) and heated to 95° C. for 60 min, cooled, mixed with fungal spores (Rhizopus oligosporus), vacuumed and filled into cartridges. A 20×20×20 mm object is layered with 50% gas phase by rotating the substrate strands 90° to each other from layer to layer and arranging the strands in a regular pattern so that the mean distances between strands are equal. The object is incubated for 72 h at 25° C. and a relative humidity of 90%, then packed, vacuumed and stored in a cool place or frozen.

In another exemplary embodiment, okara is pressed with a dry matter of 18%, the dry matter is adjusted to 28%, soybean oil (5%, w/w) is added and heated to 95° C. for 60 min, cooled, fungal spores added, vacuumed and filled into cartridges. An oval object of the size 100×50×18 mm is produced layer by layer with a gas phase content of 25% by oscillating the substrate strands always oriented in one direction, horizontally with respect to position, in such a way that the strands touch each other from layer to layer at the maximum point of oscillation. The object is incubated for 72 hours at 25° C. and a relative humidity of 90%. After fermentation is complete, soy milk, concentrated to a dry matter of 25%, is used to fill the cavities and gelled with GDL at 25° C. for 5-10 h. After gelation, the object is then packed, vacuumed, and stored in a cool place or frozen.

The fermentations preferably take place at temperatures between 15 and 40° C. with a relative humidity of 20-99%. In the case of fermentation with a fungus or a combination of fungi or in the case of co-fermentation with microorganisms such as lactic acid bacteria, the fermentation temperature is selected in such a way that additionally to best possible growth, sporulation is avoided.

Possible fungal species for fermentation are filamentous fungi of the genus Rhizopus (for example Rhizopus oligosporus, Rhizopus stolonifer, Rhizopus oryzae, Rhizopus arrhizus), Actinomucor elegans (typically used for the production of Meitauza), Aspergillus oryzae (typically used in the production of soy sauce), Bacillus natto (typically used in the production of natto), Neurospora intermedia (typically used in the production of “oncom” or “ontjom”, which means fermented peanut press cake). The accompanying bacterial flora can also contribute to the fermented end products naturally having nutritional benefits, for example, increased—vitamin content or better digestibility (Rhizopus oligosporus, Aspergillus-oryzae). Possible bacterial species for fermentation are, for example, lactic acid bacteria (e.g. Lc. Lactis, Lb. Bulgaricus, Prop. bact. freudenreichii, Lb. Reuteri) or Zymomonas mobilis).

A fermentation in the sense of the invention requires at least one fermentation with a mycelium-forming fungus, but may contain further mycelium-forming or non-mycelium-forming fungi as well as non-mycelium-forming microorganisms. The fermentations may be carried out as singular fermentations and as co- or multiple fermentations. Fungi are used, for example, for structuring (formation of a microstructure), enrichment with nutritionally valuable compounds/substances (e.g., vitamins), modulation of digestibility, and flavor formation (e.g., by degrading undesirable compounds or by segregating sensory beneficial compounds or by providing substances that can be further utilized by other fermentation cultures), microorganisms are used, for example, for flavor formation (e.g., for degrading undesirable sensory beneficial compounds), segregation of sensory beneficial compounds, combinations thereof, provision of substances that can be further utilized by other fermentation cultures), structuring (for example by changing the pH value, cross-linking of structures within the substrate), partial degradation of the substrate and/or release of substances modulating fungal growth by degradation and/or excretion, alteration of visual aspects (e.g., color), enrichment with nutritionally beneficial compounds/substances (e.g., vitamins), modulation of digestibility or improved shelf life. Overall, the combinations are primarily chosen so that the sensory properties (taste, texture) can be tailored. In co- or multiple fermentation, the organisms can complement each other functionally or work synergistically with each other. A suitable combination of co- or multiple fermentations can produce meat-like sensory properties (especially chicken flavor in terms of taste and a fibrous texture/structure).

In another embodiment, the fungal spores are distributed anisotropically in the object, such that the substrate contains significantly fewer spores at some locations in the object than at other locations. The degree of anisotropy is achieved, for example, by combining a spore-free or spore-poor phase and a spore-rich phase in one object by extruding two phases separately. In another embodiment, the spores are anisotropically distributed within the substrate strands by co-extruding two substrate phases—a spore-free or spore-poor phase and a spore-rich phase—in such a way that the concentration of spores is preferably increased in the outer phase compared to the inner phase.

In a further embodiment, inoculation with spores is carried out after extrusion and construction of the object either by spraying the interfaces with a spore-containing atomized liquid or by wetting the entire object by immersion in a spore-containing fluid, especially if a preceding step has exceeded a temperature critical for spore vitality. Anisotropic distributions are also achieved when products are inoculated with more than one type of fungal spore, the spores are placed in separate substrate masses and localized differently in the object.

One of the characteristics of the products produced is that the mechanical strength of the products usually increases over the fermentation time. Further preparation usually changes the firmness of the objects towards lower strengths, whereby the extent of the reduction depends on the preparation method. In a preferred embodiment, the objects remain dimensionally stable when boiled in water or cooked in steam and do not expand.

In one possible embodiment, the substrate phase is arranged either free-standing or with the aid of devices in such a way that a tube-like structure is created, bounded by the substrate phase in two spatial directions and unlimited in the third dimension. In a first step, mycelium is formed, and the substrate phase is solidified by fermentation, and in a further step, a fluid either flows through or fills the tubular structure. In a subsequent fermentation, the composition or the chemical and physical properties of the flowing or standing fluid are modified by interaction with the fungal mycelium and/or interaction with the substrate phase. The fungal mycelium is supplied with oxygen via the outer side of the tube formed by the substrate phase. The formation of the tube can be supported by the application of a perforated material, which on the one hand allows oxygen supply to the fungal mycelium inside the tube, but on the other hand also reduces or prevents leakage of the fluid in the tube and gives the whole construct a minimum of strength. After fermentation, the fluid is separated, filtered, and dried.

In another embodiment, a random arrangement of the substrate phase is selected, the object is fermented and then completely filled with a fluid and fermented for a further period of time. After fermentation, the fluid is separated, filtered. and dried. Such a fluid is characterized by the fact that the enzymatic activity of the mycelium has caused, among other things, a partial hydrolysis of the proteins and thus a sensory change.

After fermentation, the fermentation objects or objects produced specifically for this purpose can again be subjected to a partial or full de-structuring step. For example, a rather coarsely comminuted object can represent the basic mass for a meat patty, a rather finely comminuted object as well as a rather or very coarsely comminuted object can serve as mass for a subsequent wet extrusion, in a wide temperature range.

A process is thus conceivable for the production of a structured body which, for the purpose of and during fermentation, is permeated with unfilled cavities, is solid and fermented, which is formed on the basis of modulable masses, wherein

(a) at least one rheologically and texturally adjustable, modulable mass, which builds up the body, forms a directed or undirected mesostructure, which is freely adjustable with regard to its arrangement in certain areas and which forms the substrate as well as the cavities for one or more fermentations and a basic structure which is decisive for the overall texture, and
(b) by the introduction of at least one co- and/or superimposed microstructure induced by one or more fermentations in such a way that partially or completely coherent filamentary (fungal growth) network structures are produced in, on and between the mesostructural elements; and
(c) that by choosing the volume fraction of unfilled compartments in the whole object, by choosing their arrangement, and by choosing the ratio between mesostructure surface area and mesostructure volume, the growth of the mycelium as a whole and the penetration and direction of the mesostructure with mycelium
(d) and thus in their entirety the network structures are adjustable and that the totality of the structuring elements at the micro and meso level in their interplay bring about an adjustable (i) solidification, (ii) rheological properties and (iii) sensory-relevant texturization.

The substrate phase can be produced by directional or non-directional 3D extrusion.

The partially or completely coherent, filamentous, fungal wax network-forming fermentation comprises one or more fungal cultures and a volume fraction of the volume of the unfilled cavities of at least 0.1%.

The mycelium forming spores are present in a mass and isotropically distributed in the typical cross-section of the mesostructural elements formed therefrom, and/or (ii) are predominantly concentrated in the outer 40% (v/w) of the mesostructural elements, and/or (iii) the distribution of the spores in the typical cross-section of the mesostructural elements formed exhibits a gradient from the center towards the location of maximum distance from the center, or (iv) at least 95% is found on the surface of the mesostructural elements.

The masses used have a lipid content of 0-70% (w/w), a protein content measured as nitrogen of 0-50% (w/w) and a carbohydrate content of 0-80% (w/w) as well as other ingredients.

In the drawing, the invention is illustrated—partly schematically—by means of embodiments: It shows:

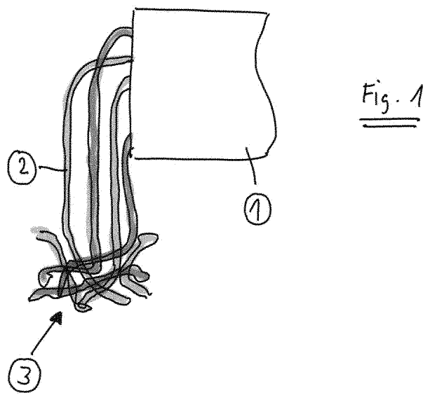

FIG. 1 shows the exit of several extruded matrix strands from an extruder, partly broken off;

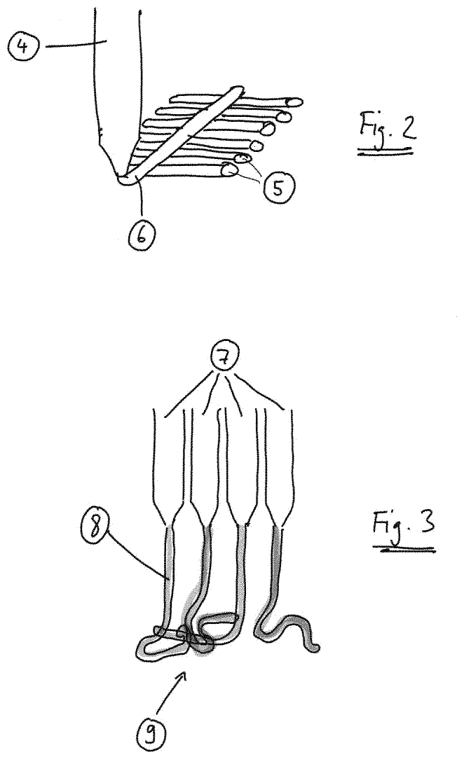

FIG. 2 shows the application of extruded matrix strands to another strand layer by means of a nozzle, broken off;

FIG. 3 shows several extrusion nozzles connected in parallel;

FIG. 4 a perforated plate with variously shaped openings from which strands of material can emerge;

FIG. 5 an extrusion device with different extrusion nozzles and a motor-driven, rotating knife downstream in the conveying direction of the extruded strands;

FIG. 6 a chaotic heap of extruded material strands;

FIG. 7 strand pieces, chaotically arranged;

FIG. 8 cut strands;

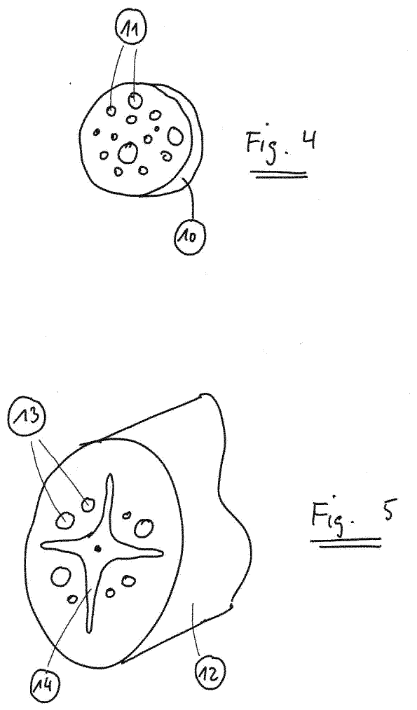

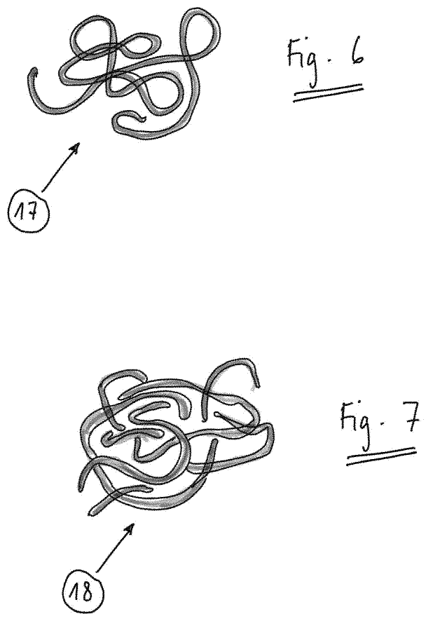

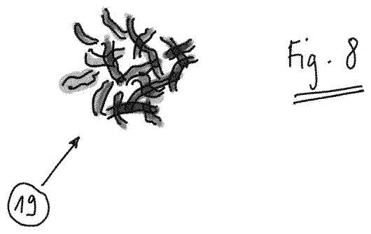

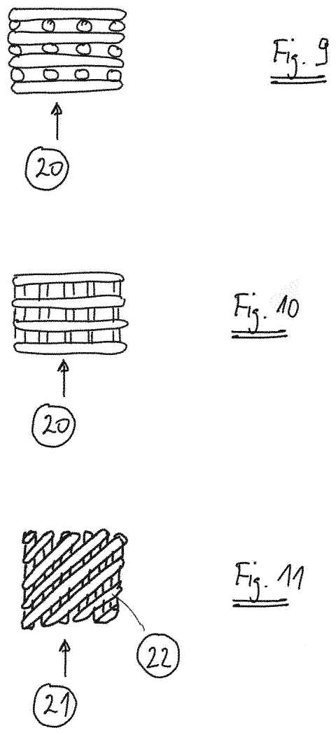

FIG. 9 side view of a matrix body consisting of extruded material strands arranged at right angles to each other in different planes;

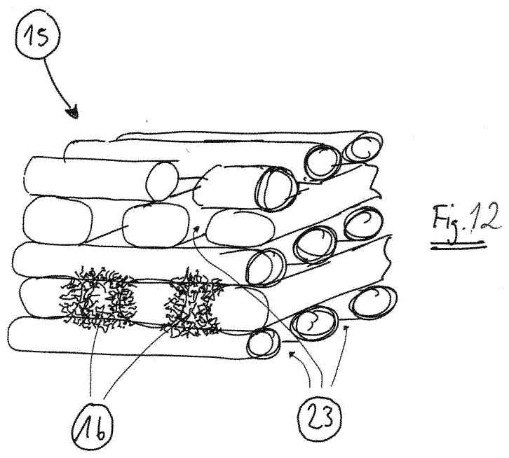

FIG. 10 top view of the embodiment shown in FIG. 9;

FIG. 11 another embodiment in which the material strands are arranged at angles to each other;

FIG. 12 a representation similar to FIG. 9 on a larger scale, shown in perspective;

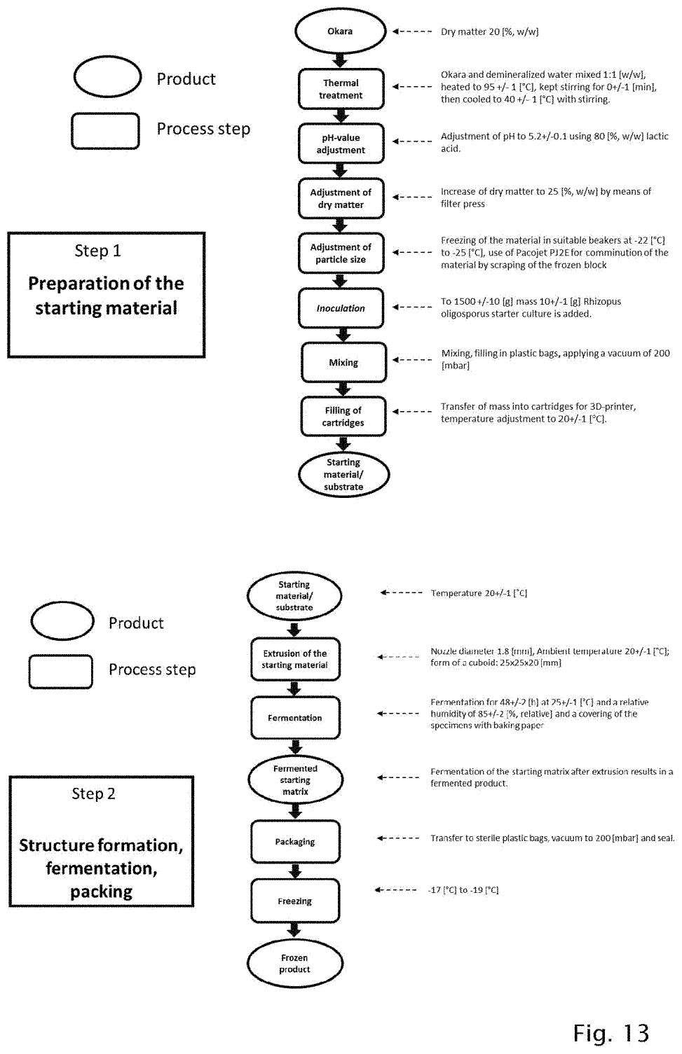

FIG. 13 preparation of the starting material and formation of the starting matrix, fermentation, packaging

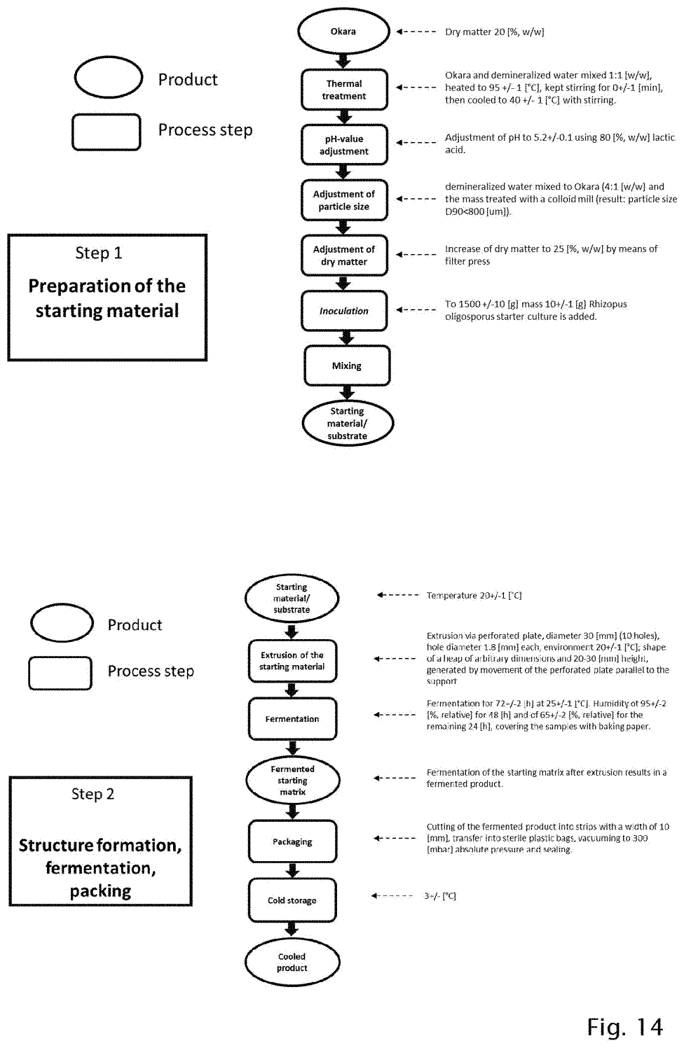

FIG. 14 preparation of the starting material and formation of the starting matrix, fermentation, packaging;

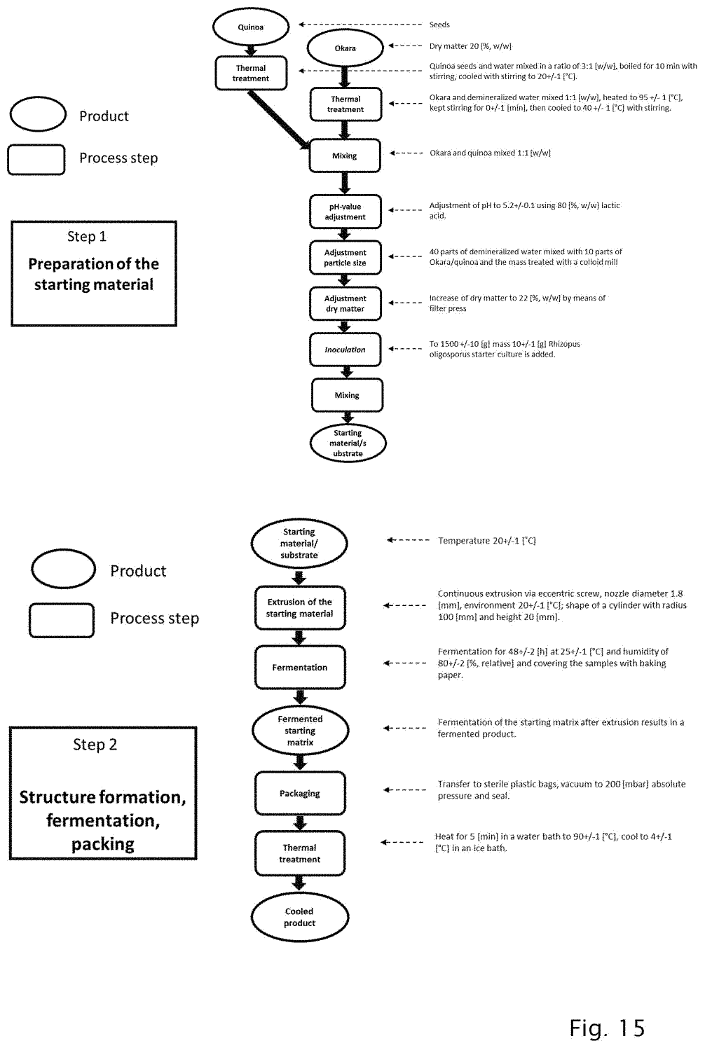

FIG. 15 preparation of the starting material and formation of the starting matrix, fermentation, packaging

The reference sign 1 shows a part of an extruder that has several outlet nozzles, not shown in detail, from which, in the embodiment shown, a total of four product strands 2 emerge, which combine under the influence of gravity to form a chaotic heap 3. Depending on the requirements, the product strands 2 can be subdivided, in particular cut off, in a time- or volume-controlled manner, after which the heap 3 is transported away intermittently.

In the embodiment shown in FIG. 2, a nozzle 4, which may be motor-driven, is shown, with several product strands 5 arranged parallel to and at a distance from each other. In the embodiment shown, the nozzle 4 ejects the product strand 6 at right angles to the longitudinal axis of the product strands 5. Several such layers of product strands 5, 6 can be arranged one above the other and/or next to each other and complement each other to form an object.

In the embodiment according to FIG. 3, four nozzles 7 are arranged parallel and at a distance from each other and are assigned to an extruder not shown, from which product strands 8 emerge and are separated, for example, in a time-controlled or volume-controlled manner. The product strands 8 can combine to form a chaotic heap 9 or be combined in some other way to form a product body.

FIG. 4 shows a perforated plate 10 that is assigned to an extruder that is not shown. The perforated plate 10 has outlet openings 11 of different diameters from which product strands emerge.

In the illustration in FIG. 5, 12 shows a part of an extruder that has nozzles 13 of the same or different diameters from which product strands emerge. Downstream in the conveying direction is a rotating knife 14 which cuts the product strands. These can then be transported away individually or arranged at any angle to each other to form a body.

FIG. 12 shows a product 15 consisting of several layers of product strands arranged one above the other. In the embodiment shown, a fungal mycelium 16 grows in the cavities. For reasons of simplification, this is only shown in two places. Of course, the corresponding fungus grows in the various cavities of the product body 15.

FIG. 6 shows another chaotic heap 17 consisting of a practically endless strand, while FIG. 7 shows a heap 18 consisting of divided product strands. In FIG. 8, the heap 19 consists of divided product strands.

FIGS. 9, 10 and 11 show different product bodies. For example, the product body 20 in FIG. 9 has a similar structure to the product body in FIG. 12 and consists of several layers of product strands each running at right angles to each other with their longitudinal axes, which also applies to the embodiment according to FIG. 10, while in the embodiment according to FIG. 11 the product body 21 consists of product strands 22 running at an acute angle to each other. However, the angles can also be different from layer to layer.

The features described in the claims and in the description, as well as those apparent from the drawing, may be essential to the realization of the invention either individually or in any combination.

### LIST OF REFERENCES

1 Extruder
2 Product strand, strand, matrix strand, mesostructure element, structuring element
3 Heap
4 Nozzle
5 Product strand, strand, matrix strand, mesostructure element, structuring element
6 Product strand, strand, matrix strand, mesostructure element, structuring element
7 Nozzle
8 Product strand, strand, matrix strand, mesostructure element, structuring element
9 Heap
10 Perforated plate
11 Outlet opening
12 Extruder
13 Nozzle
14 Knife
15 Product, product body, body
16 Fungus, fungal mycelium, microorganism, network structures
17 Heap
18 Heap
19 Heap
20 Product body, product, body
21 Product body, product, body
22 Product strand, strand, matrix strand, mesostructure element, structuring element
23 Channels, pores, cavities, unfilled cavities

### BIBLIOGRAPHY

[1] Heine, D., Rauch, M., Ramseier, H., Müller, S., Schmid, A., Kopf-Bolanz, K., Eugster, E. (2018). Plant proteins as meat substitutes: a review for Switzerland. Agrarforschung Schweiz 9(1), 4-11.
[2] Bio Suisse—Guidelines for the production, processing and trade of Bud products. Version as of 1 Jan. 2019. Link: https://www.bio-suisse.ch/media/VundH/Regelwerk/2019/DE/rl_2019_1.1_d_gesamt_11.12.2018.pdf
[3] O'Toole, D. K. (2004). Soymilk, Tofu, and Okara. In: Encyclopedia of Grain Science (2004). Edited by Wrigley, C. W., Corke, H., and Walker, C. E. Academic Press.
[4] Shurtleff, W., and Aoyagi, A. (1979). The Book of Tempeh. A Cultured Soyfood.
[5] Zieger, T. (1986). Experiments on the production of tempe gembus and meidouzha. Diploma thesis carried out at the Institute for Food Technology of the University of Hohenheim.
[6] GB 1 277 002 A
[7] U.S. Pat. No. 3,885,048 A
[8] GB 2 007 077 A
[9] DATABASE GNPD [Online] MINTEL; 27. Mai 2015, anonymous: «Light Manioc Pasta”, XP055633351

### SUMMARY

The invention relates to a process for producing a product from one or more biological substances or mixtures thereof. Furthermore, the invention relates to a product manufactured according to the process of the invention. Furthermore, the invention relates to the use of such a product.

1.-34. (canceled)

35. Process for producing a product from one or more biological substances or mixtures thereof, which, optionally after cleaning, after adjustment of the dry matter, optionally subsequent thermal treatment such as boiling, for example, as well as comminution and optionally further pre-processing to change the material properties and/or nutritional physiological properties of the starting material, are extruded, and through the extrusion process a strand or strands are arranged into a starting matrix that has channels, pores or cavities that are completely or partially open to the outside, and within which or between which one or more fungus/fungi and optionally additional fermenting microorganism(s) grow that before, during, or after the extrusion process are introduced or applied into or onto the starting matrix in the form of the vegetative or permanent form, and the fungus/fungi cross-link with the starting matrix and/or grow in this while the starting matrix is subject to a fermentation process or co-fermentation process, and the cross-linking and/or growth of the fungus/fungi decisively influences and/or partially influences the texture and/or firmness, and that the product produced from the starting matrix is if needed subsequently cut into pieces of predetermined dimensions, packaged, and supplied for further purposes of use whereby in the case of edible products, the taste and/or texture is determined by the fungus/fungi growing in the pores, channels and/or cavities and/or by other microorganism(s) introduced into the pores, channels, cavities and/or into the starting material(s) and/or by the duration and/or the temperature profile of the fermentation process and/or by the adjustment of the water content of the product during or after fermentation and/or by the composition of the biological starting material and/or by the volume fraction of pores, channels, cavities in the starting matrix and/or by the arrangement of the pores, channels, cavities and/or by the quantity of the interface between the totality of the strands and the totality of the pores, channels, cavities and/or by the diameter(s) of the strands and/or by gas exchange with the environment and/or by a process-technical pre-treatment adjusting the rheology of the starting material whereby the nozzle or nozzles and the support on which the starting material(s) is discharged from the nozzle or nozzles is/are movable relative to one another, so that either a chaotic distribution of the discharged matrix strands, forming a random heap, or a predetermined distribution of the discharged matrix strands in predetermined angular arrangements (for example 20, 21) relative to one another is effected.

36. Process for the production of a structured body that is permeated with unfilled cavities (open channels, pores or cavities), solid, fermented, and is formed on the basis of modulable substrates (synonymous with starting material), wherein
(a) at least one rheologically and texturally adjustable, modulatable mass forms a directional or non-directional mesostructure (synonymous with starting matrix) that is freely adjustable in terms of arrangement over a wide range and that forms the substrate (synonymous with starting matrix) as well as the cavities (synonymous with pores, channels, cavities) for one or more fermentations and a basic structure (synonymous with starting matrix) that is co-decisive for the overall texture;
(b) by the introduction of at least one co- and/or superimposed microstructure (synonymous with fungal mycelium or network structure) induced by one or more fermentations, such that partially or completely interconnected filamentous network structures (synonymous with microstructure) caused by fungal growth are produced in, on and between the mesostructure elements (synonymous with extruded strands or strand pieces);
(c) by choosing the volume proportion of unfilled cavities, channels or pores, compartments in the total object, by choosing their arrangement as well as by choosing the ratio between mesostructure surface and mesostructure volume, the growth of the mycelium as a whole as well as the penetration and direction of the mesostructure with mycelium and thus in its entirety the network structures are adjustable and
(d) the totality of the structuring elements at micro and meso level interact to effectuate an adjustable (i) solidification, (ii) rheological properties and (iii) sensory-relevant texturing.

37. Process according to claim 35, wherein by the extrusion process simultaneous and/or in parallel and/or sequential extrusion process steps a body as a starting matrix is prepared, which consists of several extruded strands lying above and/or next to and/or behind one another, which are connected materially or functionally to connect into one unit at their mutually touching surfaces and form cavities, channels or pores between them, into which the fungus/fungi arrange/s itself/themselves.

38. Process according to claim 35, wherein the growth and/or metabolic activity of the fungus/fungi can be interrupted and/or changed and/or controlled after a specific period of time for the respective starting material.

39. Process according to claim 35, wherein the previously empty pores, channels or cavities occupied by the fungal mycelium/fungal mycelia after fermentation are partially or completely filled with a flowable and/or partially or completely solidifying material, wherein the solidification is carried out via an additional fermentation with the aid of a further bioactive organism, enzymatically, via thermo-reversible mechanisms, ionically induced, by heating or by other processes.

40. Process according to claim 35, wherein the starting material for the starting matrix is processed by means of extrusion, co-extrusion or multi-extrusion processes to form a product strand and/or product strands and/or product strand pieces, the product strand subsequently remaining as a continuous strand or disintegrating into individual pieces and/or being disintegrated, and the temperature of the product strand and/or of the product strands and/or of the product strand pieces immediately at the die or perforated plate outlet being 2 to 99.5 [° C.], preferably from 5 to 99 [° C.], more preferably from 7 to 80 [° C.], more preferably from 10 to 70 [° C.], more preferably from 12 to 60 [° C.], more preferably from 12 to 45 [° C.], most preferably from 15 to 25 [° C.].

41. Process according to claim 35, wherein the extruded strand or strand pieces are foamed with gas inclusions caused by expansion of a compressed gas, e.g. CO₂, N₂O, O₂, or by gas formation in the course of fermentation, e.g., CO₂, by foaming of the material before discharge into the product, e.g., with CO₂, O₂, N₂, air, or by a chemical reaction, e.g., that of a carbonate with an acid, or by the expansion of water to water vapor within the strands or strand pieces.

42. Process according to claim 35, wherein the fungi/fungus spores/molds/mold spores used for the fermentation originate from the genus Rhizopus, for example Rhizopus oligosporus, Rhizopus stolonifer, Rhizopus oryzae, Rhizopus arrhizus and/or from the genus Actinomocur, for example Actinomocur elegans spp. meitauza and/or from the genus Aspergillus, for example Aspergillus oryzae and/or from the genus Penicillium, for example Penicillium candidum, Penicillium camemberti, Penicillium roqueforti, Penicillium glaucum, and/or from the genus Geotrichum, for example Geotrichum candidum, and/or of another genus capable of—modifying the texture and/or sensory characteristics of the product, as well as the microorganism(s) used for the microbial fermentation or co-fermentation from the genus Bacillus, for example Bacillus subtilis spp. natto and/or from the genus Neurospora, for example Neurospora intermedia and/or from the genus Lactobacillus, for example Lactobacillus bulgaricus, Lactobacillus reuteri and/or from the genus Lactococcus, for example Lactococcus lactis and/or from the genus Propionibacterium, for example Propionibacterium freudenreichhii and/or from the genus Zymomonas, for example Zymomonas mobilis and/or from the genus Leuconostoc, for example Leuconostoc mesenteroides and/or from another genus which is suitable for changing the texture and/or sensory properties of the product.

43. Process according to claim 35, wherein the inoculation of the starting matrix with fungal mycelium and/or fungal spores and/or mold mycelium and/or mold spores is carried out in such a way that they are, for example, mixed with the starting material and/or sprayed onto the starting matrix and/or the product is soaked in and/or with a suspension of the fungal mycelium and/or the fungal spores and/or the mold mycelium and/or the mold spores.

44. Process according to claim 35, wherein the starting material is conveyed in the extrusion process through nozzles or openings, such as in a perforated plate, with an inside diameter of 0.4 to 9 [millimeters], preferably 0.5 to 7 [millimeters], preferably 0.8 to 5 [millimeters], preferably 1 to 3.5 [millimeters], again preferably between 1 and 2.5 [millimeters], in particular 1.1 to 2 [millimeters], the diameters of the openings having the same or different diameters in the case of parallel or consecutive extrusion processes.

45. Product according to claim 35, wherein in the case of edible products, the taste and/or texture is determined by the fungus (i) introduced into the pores, channels and/or cavities and/or by further microorganisms introduced into the pores, channels, cavities and/or into the starting material(s) and/or by the duration and temperature profile of the fermentation process and/or by the adjustment of the water content of the product during or after fermentation and/or by the composition of the biological starting material and/or by the volume fraction of pores, channels, cavities in the starting matrix and/or by the arrangement of the pores, channels, cavities and/or by the quantity of the interface between strands and pores, channels and cavities and/or by the diameter(s) of the strands and/or by gas exchange with the environment and/or by a technical pre-treatment process adjusting the rheology of the starting material.

46. Product according to claim 45, wherein several layers or sheets of extruded product strands are arranged in predetermined or chaotic angular arrangements above and/or next to and/or behind each other.

47. Product according to claim 45, wherein the starting material for the starting matrix consists of biological substances or mixtures thereof that allow and/or promote a desired germination and/or growth of the fungus/fungi or their spores/permanent forms and optionally of the microorganism(s) or their permanent forms due to the material composition as well as the adjusted dry matter content and/or further suitable treatment steps, such as, for example, biological substances with increased protein content in the dry matter, such as, for example, peas, soy, quinoa, chickpeas, tofu, seitan, cream cheese masses, processed cheese masses, ricotta and/or with increased fiber content in the dry matter, such as okara, spent grains, whole grain cereal products, largely insoluble residues from fat/protein extraction—and/or increased fat content in the dry matter, such as almonds, cashew, soy and/or high carbohydrate content in the dry matter, such as wheat or other cereals or pseudo-cereals and/or hydrocolloids such as gels based on gelatin, pectin, starch and/or pastes such as concentrated dispersions of any powders such as milk protein, whey protein isolate or plant-based protein concentrates or isolates, whereby in each case the adjustment of the water content is effected in such a way that the substances have a yield stress.

48. Product according to claim 45, wherein the proportion of cavities, channels or pores is 20 to 85 [%, volume fraction], preferably 20 to 75 [%, volume fraction], again preferably from 25 to 75 [%, volume fraction], again preferably between 25 and 70 [%, volume fraction], in particular preferably between 25 and 60 [%, volume fraction], most preferably between 30 and 55 [%, volume fraction].

49. Product according to claim 45, wherein the firmness of the product measured after fermentation compared to the firmness of the underlying starting matrix before fermentation increases by at least a factor of 20, preferably by at least a factor of 12, more preferably by at least a factor of 8, even more preferably by at least a factor of 5, even more preferably by at least a factor of 3.5, even more preferably by at least a factor of 2, even more preferably by at least a factor of 1.5, more preferably by at least a factor of 1.2, most preferably by at least a factor of 1.1, wherein the firmness is determined as the maximum force with a penetration measurement by means of a flat, round cylinder geometry with a diameter of 8 millimeters, which penetrates at a speed of 0.5 cm per second into a product body of the dimension 20 millimeters×20 millimeters×20 millimeters with a penetration depth of 10 millimeters at room temperature.

50. A meat substitute comprising the product according to claim 45.
